(19) United States
(12) Patent Application Publication
(10) Pub. No.: US 2025/0360170 A1
(43) Pub. Date: Nov. 27, 2025

(54) METHODS OF ISOLATING AND DELIVERING N-CADHERIN POSITIVE MESENCHYMAL STEM CELLS FOR TREATMENT OF OSTEOARTHRITIS

(71) Applicants: University of Kansas, Lawrence, KS (US); Stowers Institute for Medical Research, Kansas City, MO (US)

(72) Inventors: Jinxi Wang, Overland Park, KS (US); Mingcai Zhang, Olathe, KS (US); Rupal Soder, Overland Park, KS (US); Qinghua Lu, Overland Park, KS (US); Linheng Li, Overland Park, KS (US)

(21) Appl. No.: 19/215,109

(22) Filed: May 21, 2025

Related U.S. Application Data
(60) Provisional application No. 63/650,570, filed on May 22, 2024.

(51) Int. Cl. A61K 35/28; A61P 19/02; C12N 5/00; C12N 5/0775

(52) U.S. Cl. CPC A61K 35/28; A61P 19/02; C12N 5/0081; C12N 5/0668; C12N 2501/599; C12N 2531/00

(57) ABSTRACT

A method of sorting for N-cadherin positive (N-cad+) cells may comprise providing a sample of cells having N-cad+ cells and N-cad-cells. The cells may be incubated with a N-cadherin antibody (biotinylated). The cells may be incubated with a coupling substrate (anti-biotin substrate). Cells not bound to the N-cadherin antibody may be removed. The N-cad+ cells may be collected. The sample may include Wharton's jelly mesenchymal stem cells (WJMSCs). The coupling substrate may include magnetically-responsive microbeads. A magnetic field may be applied to the cells. A method of treating joint disease in a subject may comprise providing a subject having joint disease. N-cad+ cells may be introduced into a joint space of the subject. The N-cad+ cells may be injected into an intraarticular space via at least two injections at an angle with respect to each other.

### CROSS-REFERENCE TO RELATED APPLICATIONS

This patent application claims priority to U.S. Provisional Application No. 63/650,570 filed May 22, 2024, which provisional is incorporated herein by specific reference in its entirety.

### BACKGROUND

### Field

The present disclosure relates to collection of N-cad+ cells for use as treatment for a joint disease, such as osteoarthritis.

### Description of Related Art

Osteoarthritis (OA) is a prevalent joint disease that affects over 32 million American people and costs the United States more than $60 billion per year. Current clinical managements of OA are used mainly for symptomatic control. There are no pharmacologic therapies available to permanently halt or reverse the progression of OA. Once severe OA has developed, joint replacement and joint fusion are the only surgical options, which are not desirable for younger patients due to a limited lifespan of prosthetic joint implants. Transplantations or intra-articular (IA) injections of bone marrow, adipose, and umbilical cord derived mesenchymal stem cells (MSCs) into OA joints have shown some short-term symptomatic relief in preclinical and clinical trials, but their long-term outcomes remain unknown.

Previous studies showed that N-cadherin (N-cad), a member of calcium dependent transmembrane adhesion proteins, is a key factor for mediating cell-cell interactions in chondrogenesis, and that N-cad positive (N-cad+) stromal cells in the endosteal/articular region have a potential to give rise to osteoblasts, chondrocytes, and adipocytes. Upon joint injury, endogenous N-cad+ stromal cells can differentiate to chondrocytes to repair damaged articular cartilage (AC) in mice.

### SUMMARY

In some embodiments, the present disclosure provides a method of sorting for N-cadherin positive (N-cad+) cells. This method may include providing a sample of cells having N-cad+ cells mixed with non-N-cad+ cells. The cells may be incubated with a N-cadherin antibody having a coupling region (e.g., biotinylated N-cadherin antibody). The cells may be incubated with a coupling substrate (e.g., anti-biotin substrate) that binds with the coupling region to form a coupling region and coupling substrate connection. Cells not bound to the N-cadherin antibody having a coupling region (e.g., biotinylated N-cadherin antibody) may be removed. The N-cad+ cells bound to the N-cadherin antibody having a coupling region (e.g., biotinylated N-cadherin antibody) may be collected. In some aspects, the a coupling region and coupling substrate coupling are any coupling pair of entities or moieties that couple with each other.

Another embodiment of the present disclosure provides another method of sorting for N-cadherin positive (N-cad+) cells. This method may include providing a sample of cells having N-cad+ cells mixed with non-N-cad+ cells. The cells may be dissociated. The dissociated cells may be shaken. The shaken cells may be filtered. Dead cells may be removed from live cells of the cells. The live cells may be optionally applied to a separation column system capable of providing a magnetic field. Flow-through cells may be collected. The cells may be incubated with a biotinylated N-cadherin antibody. Unbound antibody may be washed off. The cells may be incubated with an anti-biotin substrate. Cells may be washed to remove anti-biotin substrate that is not bound to the biotinylated N-cadherin antibody. Cells not bound to the biotinylated N-cadherin antibody may be removed. N-cad+ cells bound to the biotinylated N-cadherin antibody may be collected.

Another embodiment of the present disclosure provides a method of treating a joint disease (e.g., osteoarthritis) in a subject. This method may include providing the subject having osteoarthritis with N-cadherin positive (N-cad+) cells, wherein the cells may include human umbilical cord derived Wharton's jelly mesenchymal stem cells (WJMSCs) and the N-cad+ cells may be N-cad+ WJMSCs. The N-cad+ cells may be introduced into a joint space of the subject.

Yet another embodiment of the present disclosure provides a method of preparing

a hydrogel having N-cad+ cells and may include preparing a hydrogel capable of passing through 27 G needle or other a gauge needle. In some embodiments, N-cad+ cells may be combined with the hydrogel for administration into a joint space of a subject having a joint disease. According to some embodiments, the present disclosure provides, an isolated cell population prepared by the process of: providing a sample of cells having N-cad+ cells mixed with N-cad− cells; dissociating the cells in the sample; removing dead cells from the dissociated cells to obtain live cells; incubating the live cells with a binding molecule that binds to N-cadherin; and selecting N-cad+ cells using the binding molecule to produce the isolated cell population. In some embodiments, the binding molecule is a biotinylated antibody. In some embodiments, the N-cad+ cells are selected by contacting the biotinylated antibody with an anti-biotin substrate.

In some embodiments, the isolated cells are effective to treat a joint disease. In some embodiments, the isolated cells are MSCs. In some embodiments, the isolated cells are MSCs obtained from Wharton's jelly. In some embodiments, the isolated cells are human cells. In some embodiments, the isolated cells produce elevated levels of anti-inflammatory cytokines relative to a native population of cells. In some embodiments, the isolated cells are effective to suppress the expression of catabolic and inflammatory genes in articular cartilage.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes a schematic showing the isolation process of N-cad+ and N-cad−hWJMSCs using a magnetic labeling or magnetic-activated cell sorting (MACS) procedure with a magnetic biotinylated N-cadherin specific antibody.

FIG. 2 includes representative photomicrographs that show various levels of OA severity in the rat knee joints at 4 weeks after receiving different IA treatments or at 8 weeks after the destabilization of the medial meniscus (DMM) surgery that induces OA.

FIG. 3 includes a scatter plot that shows the distribution of OA score points for each of the 8 treatment groups.

FIG. 4 illustrates a one-angle IA injection at a 75° angle versus a two-angle (90° and) 45°) IA injection.

FIG. 5. includes photomicrographs showing the difference in tissue adhesion and staining quality before and after the modification of the method for processing tissue sections and staining procedure.

FIG. 6 shows the relative expression fold change of the Acan, Col2a1, Il-10, Il-1b (e.g., Il-1β), Tnf-α (e.g., Tnf-α), and Adamts5 genes in the cartilage of rat osteoarthritic knee joints treated with AD, BM, MWJ, Ncd+WJ and Ncd−WJ cell groups. The gene expression level of the PBS group has been normalized to 1.0.

FIG. 7 includes the relative fold change of the Prg4, CD163, FoxP3, Il-1β, Tnf-α, and Adamts5 genes in the synovium of rat osteoarthritic knee joints treated with the same cell groups. The gene expression level of the PBS group has been normalized to 1.0.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented herein.

Generally, the present disclosure relates to systems and methods for sorting N-cadherin positive (N-cad+) and N-cadherin negative (N-cad−) MSCs from human umbilical cord tissue, to obtain human Wharton's jelly-derived MSCs (hWJMSCs). Then, the N-cad+ hWJMSCs cells can be delivered for therapeutic purposes into a subject in need of a therapy for a joint disease. The joint disease may be osteoarthritis (OA) or an injury or other indication.

In some embodiments, the system and method for selecting the N-cad+ hWJMSCs as described herein is an improvement that allows for high performance sorting that can be performed on an industrial scale. In some embodiments, the sorting of the N-cad+ hWJMSCs can be performed to remove dead cells. In some embodiments, the sorting can be performed without using fluorescence-activated cell sorting (FACS). In some embodiments, the sorting of the N-cad+ hWJMSCs can be performed with magnetic-activated cell sorting (MACS). In some embodiments, the sorting results in lower cell loss, higher yield of sorted cells, and 4-6 times shorter sorting time than FACS.

In accordance with some embodiments of the present disclosure, an exemplary method of sorting for N-cadherin positive (N-cad+) cells can include providing a sample of cells having N-cad+ cells mixed with non-N-cad+ cells. The sample of cells can be from a natural tissue sample, or a laboratory mixture of cells. The sample of cells may include Wharton's jelly mesenchymal stem cells (WJMSCs) and the N-cad+ cells may be N-cad+ WJMSCs.

To prepare the sample of cells for subsequent processes, in some embodiments, the method may further include dissociating the cells in the sample from each other. In some embodiments, the method may include shaking the dissociated cells sufficiently to present

N-cadherin on cell surfaces of the dissociated cells. In some embodiments, the cell dissociation can also be done by using a lysing agent that lyses cell to cell linkages, such as Trypsin or other linkage cleaving enzyme. In some embodiments, the method may further include filtering the cells after the shaking. In some embodiments, the filtering may be performed with a 80-100 micron cell strainer, 60-80 micron cell strainer, or 3-60 micron cell strainer.

In some embodiments, the method may further include removing dead cells from the filtered cells to obtain live cells. In some embodiments, the dead cells may be removed from the filtered cells with dead cell removal microbeads. In some embodiments, the method may include incubating the live cells with a biotinylated N-cadherin antibody. Some embodiments provide further incubating the cells with an anti-biotin substrate. In some embodiments, the anti-biotin substrate may include microbeads. The microbeads may be magnetically-responsive microbeads. Accordingly, in some embodiments, the anti-biotin substrate can be a member that can be selectively retained.

In some embodiments, the method may include applying a magnetic field to the cells, which may include cells bound with the biotinylated N-cadherin antibody attached to the anti-biotin magnetically-responsive microbeads. The magnetic field may be a permanent magnetic field or an electromagnetic field. In some embodiments, the method may include placing the cells, which may include cells bound with the biotinylated N-cadherin antibody attached to the anti-biotin magnetically-responsive microbead, in a separation system having the magnetic field. The separation system may include a separation column associated with the magnetic field.

In some embodiments, the method may include allowing cells not bound to the biotinylated N-cadherin antibody to separate from the cells bound with the biotinylated N-cadherin antibody attached to the anti-biotin magnetically-responsive microbeads. In some embodiments, the method may include collecting N-cad− cells not bound to the biotinylated N-cadherin antibody, wherein the cells bound with the biotinylated N-cadherin antibody attached to the anti-biotin magnetically-responsive microbeads may be retained by the magnetic field. The unbound cells that are collected can be considered N-cad− cells (e.g., N-cad negative cells). Accordingly, the N-cad− cells can be stored, cultured, or otherwise retained for further use, such as in assays.

In some embodiments, the method may include removing the magnetic field from the cells bound with the biotinylated N-cadherin antibody attached to the anti-biotin magnetically-responsive microbeads. In some embodiments, the method may further include collecting the cells bound with a biotinylated N-cadherin antibody attached to the anti-biotin magnetically-responsive microbeads. In some embodiments, the method may further comprise removing the anti-biotin microbeads from the collected cells that are bound with a biotinylated N-cadherin antibody attached to the anti-biotin magnetically-responsive microbeads. In some embodiments, the method may include washing unbound biotinylated N-cadherin antibody before removing the magnetic field. In some embodiments, the method may include washing the cells after being exposed to the anti-biotin magnetically-responsive microbeads to remove any unbound anti-biotin magnetically-responsive microbeads and before collecting any of the cells.

In some embodiments, the biotinylated N-cadherin antibody can be configured with the antibody that is adapted to bind with N-cadherin. As used herein, “biotinylated antibodies” are antibodies that have been chemically conjugated with biotin that has a high affinity for binding to avidin, streptavidin, or neutravidin proteins. The process of biotinylation involves the chemical attachment of biotin molecules to the antibody, usually at the antibody's lysine residues, while preserving the antibody's ability to bind to its target antigen. In some aspects of the present disclosure, the chemical conjugation can include a cleavable linker. This allows separation of the antibody from the biotin, and thereby can release the magnetic microbeads from N-cad+ cells.

In the present disclosure, anti-biotin magnetic beads are magnetic particles that are coated with streptavidin, avidin, neutravidin, or directly with anti-biotin antibodies. These beads take advantage of the strong biotin-streptavidin interaction to capture and separate biotinylated antibodies, which may be bound to target cells or molecules.

In some embodiments, the cleavable linker between the N-cad+ antibody and the biotin is an enzyme cleavable linker. The present disclosure provides cleavable linkers for conjugating biotin to antibodies, wherein the cleavable linkers are selected from the group consisting of enzyme-cleavable linkers, pH-sensitive linkers, reducible linkers, light-cleavable linkers, and chemically cleavable linkers. Non-limiting examples of enzyme-cleavable linkers include peptide linkers that are selectively cleaved by specific proteases, such as trypsin-cleavable linkers, thrombin-cleavable linkers, or matrix metalloproteinase-cleavable linkers, as well as glycosidic linkers cleaved by glycosidases and phosphodiester linkers cleaved by phosphatases. Non-limiting examples of pH-sensitive linkers include hydrazone linkers, which are cleaved under mildly acidic conditions (pH 4-5), and cis-aconityl linkers that are cleaved at acidic pH. Non-limiting examples of reducible linkers include disulfide linkers that are cleaved in the presence of reducing agents, such as dithiothreitol (DTT) or tris(2-carboxyethyl)phosphine (TCEP), and thiol-cleavable linkers with a central disulfide bond. Non-limiting examples of light-cleavable linkers include nitrobenzyl ether linkers and o-nitrobenzyl ether linkers, which are cleaved upon exposure to ultraviolet (UV) light, as well as coumarin-based photo-cleavable linkers that are activated by specific wavelengths of light. Non-limiting examples of chemically cleavable linkers include acid-labile linkers cleaved by trifluoroacetic acid, base-labile linkers cleaved by sodium hydroxide, and metal ion-sensitive linkers cleaved by chelating agents. The cleavable linkers enable the selective and controlled release of the biotin or biotinylated antibody from the target surface, facilitating the recovery of the target antibody or cell with minimal impact on viability or functionality.

In some embodiments, the cleavable linker can be cleaved to obtain the N-cad+ cells without attachment to the magnetic particles. The method of cleaving can be determined by the type of cleavable linker used. Examples of cleavable linkers and the methods of cleaving are provided above.

In some embodiments, the present technology may include a method of treating a joint disease (e.g., osteoarthritis) in a subject. In some embodiments, the joint disease can be osteoarthritis or from an injury or any other indication. The method may include providing a subject with N-cadherin positive (N-cad+) cells, wherein the cells may include Wharton's jelly mesenchymal stem cells (WJMSCs) and the N-cad+ cells may be WJMSCs. The method may include introducing the N-cad+ cells into the joint space of the subject that is experiencing the joint disease. This allows direct treatment to the joint.

In some embodiments, the method may include injecting the N-cad+ cells into a joint space of the subject. In some embodiments, the method may include injecting the N-cad+ cells into an intraarticular space of the subject. In some embodiments, the method may include injecting the N-cad+ cells into the subject at two different angles of injection. In some embodiments, the method may include injecting a first injection of the N-cad+ cells at a first injection orientation and injecting a second injection of the N-cad+ cells at a second injection orientation that may be at an angle that is different from the first injection orientation. In some embodiments, the angle may be at least 10 degrees, at least 20 degrees, at least 30 degrees, at least 40 degrees, or at least 45 degrees. In some embodiments, the first injection orientation may be about normal (90 degrees) to about 75 degrees. The degrees can be relative to normal or relative to vertical or horizontal.

In some embodiments, the method may include combining the N-cad+ cells into a hydrogel prior to injection into the subject. In some embodiments, the method may include preparing the hydrogel to have a viscosity for injectability through a 27 G needle. In some embodiments, the method may include preparing the hydrogel to have less gel percentage compared to recommended manufacture gel percentage. In some embodiments, the gel percentage may range from 40% to 60%. In some embodiments, the hydrogel may include a hyaluronan hydrogel or a peptide-based matrix.

In some embodiments, the N-cad+ cells may be present in different amounts in a delivery composition. For example, in some embodiments, the N-cad+ cells can be present at least at 500,000 cells per microliter, or at least at 750,000 cells per microliter, or at least at 1,000,000 cells per microliter, or at least at 1,500,000 cells per microliter, or at least at 2,000,000 cells per microliter. The hydrogel may include a hyaluronan hydrogel or a peptide-based matrix.

In some embodiments, the gene expression in articular cartilage of osteoarthritic joints may be modulated by the introduction of N-cad+ WJMSCs. In some embodiments, the N-cad+ WJMSCs may promote the expression of anabolic genes such as Acan, which may encode aggrecan, a structural protein in cartilage. In some embodiments, the N-cad+ WJMSCs may also promote the expression of Col2a1, which may encode type-II collagen, a structural protein in cartilage and a marker of chondrocytes. Additionally, in some embodiments, the N-cad+ WJMSCs may promote the expression of IL-10, which may encode interleukin-10, an anti-inflammatory cytokine that may protect the body from excessive tissue damage.

Concurrently, in some embodiments, the N-cad+ WJMSCs may suppress the expression of catabolic and inflammatory genes in articular cartilage. These genes may include, but not limited to, Il-1β, which may encode interleukin-1β, a pro-inflammatory cytokine; Tnf-α, which may encode tumor necrosis factor-α, a mediator of inflammation and cell death; and Adamts5, which may encode a disintegrin and metalloproteinase with thrombospondin motifs-5, an enzyme that may cleave aggrecan and contribute to cartilage degradation.

In some embodiments, the N-cad+ WJMSCs may also modulate gene expression in the synovium of osteoarthritic joints. In some embodiments, the N-cad+ WJMSCs may promote the expression of Prg4, which may encode lubricin, a proteoglycan that may play a role in joint lubrication. In some embodiments, the N-cad+ WJMSCs may also promote the expression of CD163, which may encode a hemoglobin scavenger receptor highly expressed in M2 macrophages associated with anti-inflammatory responses and tissue repair. Additionally, in some embodiments, the N-cad+ WJMSCs may promote the expression of FoxP3, which may encode a transcription factor that may play a role in the development and function of regulatory T cells.

Similar to their effect on articular cartilage, in some embodiments, the N-cad+ WJMSCs may suppress the expression of Il-1β, Tnf-α, and Adamts5 in the synovium. This modulation of gene expression in both articular cartilage and synovium may contribute to the therapeutic effect of N-cad+ WJMSCs in treating osteoarthritis.

In some embodiments, the N-cad+ WJMSCs may demonstrate superior therapeutic efficacy compared to other types of mesenchymal stem cells, including adipose-derived MSCs, bone marrow-derived MSCs, mixed WJMSCs IMWJ), and N-cadherin negative MSCs. This superior efficacy may be reflected in the ability of N-cad+ hWJMSCs to promote the expression of more anabolic or anti-inflammatory genes and suppress the expression of more catabolic genes in both cartilage and synovium compared to other types of MSCs.

In some embodiments, methods disclosed herein can include sorting N-cad+ hWJMSCs from human umbilical cord tissue. In some embodiments, methods disclosed herein can include sorting N-cadherin negative (N-cad−) MSCs from hWJMSCs. In some embodiments, the N-cad+ hWJMSCs can be used for providing therapeutic effects from human umbilical cord derived MSCs for treatment of knee OA, or OA in any synovial joint. Also, in some embodiments, the N-cad+ hWJMSCs can be used for treatment of a joint disease or an injury in any joint.

In some embodiments, methods disclosed herein can include delivering the sorted N-cad+ hWJMSCs into knee joints, or other joint spaces. The therapeutic effect of N-cad+ hWJMSCs on OA can be significantly better than other types of MSCs that are currently used for preclinical and human clinical trials for joint disease (e.g., OA).

### Methods for Isolating Live N-cad+ and N-cad− WJMSCs

Methods for isolating N-cad+ MSCs from WJMSCs and bone marrow derived MSCs (BMMSCs) are provided. In some embodiments, a sequential procedure is used for isolating live N-cad+ and N-cad− hWJMSCs. In some embodiments, mixed hWJMSCs containing both N-cad+ and N-cad− WJMSCs can be used to show the selective sorting. In some embodiments, dead cells produced during the cell culture and expansion are removed, e.g., using a Dead Cell Removal Kit. In some embodiments, cells are incubated with a biotinylated N-cadherin antibody, e.g., at 4° for 10 min. In some embodiments, after washing off unbound primary antibody, cells are incubated with Anti-Biotin MicroBeads, e.g., at 4° for 15 min. In some embodiments, after washing off unbound microbeads, cells are applied onto a rinsed MACS LS column. In some such embodiments, after washing the column three times with 3 mL of MACS buffer, magnetically labeled cells are immediately flushed out from the column with, e.g., 5 mL of MACS buffer. In some embodiments, after Trypan-Blue exclusion staining, live N-cad+ and N-cad− cell numbers are counted using hemocytometry and the total number of live N-cad+ and N-cad− hWJMSCs are calculated and recorded.

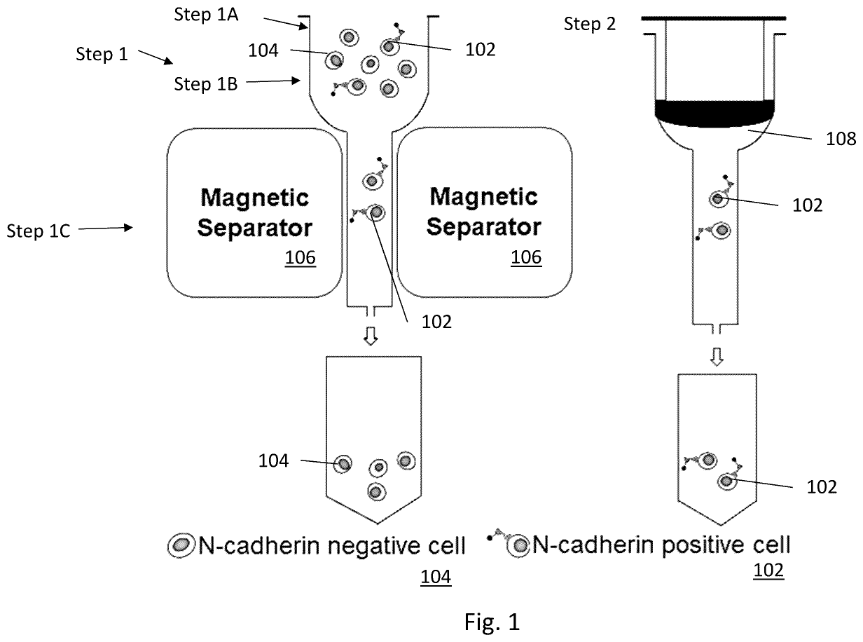

In some embodiments, the N-cad+ hWJMSCs can be produced as shown in FIG. 1. Accordingly, the isolation methods of N-cad+ and N-cad− hWJMSCs can be performed in a system that is operated over multiple steps (or sub-steps). In some embodiments, the steps that are performed can be determined on the status of the cells from the sample. Accordingly, the steps can proceed as determined by the status of the cells being processed.

FIG. 1 includes a schematic showing the isolation process of N-cad+ and N-cad− hWJMSCs using a magnetic labeling or magnetic-activated cell sorting (MACS) procedure with a magnetic biotinylated N-cadherin specific antibody. Step 1 includes Step 1A, where N-cad+ hWJMSCs 102 are labeled with the N-cadherin specific antibody, while N-cad− hWJMSCs 104 are not labeled. Then, Step 1 includes Step 1B, where the cells associated with the biotinylated N-cadherin specific antibody are incubated with anti-biotin, such as anti-biotin magnetic microbeads or other anti-biotin magnetic substrate. Then, Step 1 includes Step 1C, where magnetic separation is performed with a magnetic separator 106 that allows non-N-cad+ hWJMSCs that do not have the magnetic biotinylated N-cadherin specific antibody (e.g., other cells and N-cad− hWJMSCs 104) to pass through, while the magnetic labeled N-cad+ hWJMSCs 102 being held back by the magnetic field from the magnetic separator 106. Once the magnetic field is removed, the labeled N-cad+ hWJMSCs 102 can then be eluted in Step 2 with an elution medium 108. The magnetic microbeads can be magnetic or magnetically-responsive materials.

As used herein, “magnetically responsive material” refers to a material that reacts to an external magnetic field, such as iron particles aligning in the presence of a magnet. These materials are not necessarily magnetic themselves but can be influenced by a magnetic field. As used herein, “magnetic material” refers to a material that possesses its own permanent magnetic field, such as a magnetized piece of iron, a magnetized ferrite, or a neodymium magnet. These materials can attract or repel other magnetic materials even without an external magnetic field. For example, in some embodiments, the magnetic microbead may comprise a magnetically responsive material, wherein the magnetically responsive material is selected from the group consisting of iron, nickel, cobalt, gadolinium, dysprosium, terbium, iron alloys, nickel alloys, cobalt alloys, gadolinium alloys, dysprosium alloys, terbium alloys, carbon steel, stainless steel, iron-nickel alloys, iron-cobalt alloys, iron-manganese alloys, permalloy, mu-metal, supermalloy, neodymium-iron-boron, samarium-cobalt, magnetite (Fe₃O₄), maghemite (γ-Fe₂O₃), ferrites including strontium ferrite, barium ferrite, zinc ferrite, manganese ferrite, cobalt ferrite, ferric oxide, chromium dioxide, hematite (α-Fe₂O₃), iron oxide nanoparticles, nickel ferrite, and combinations thereof.

In some embodiments, the magnetic microbead may comprise a magnetic material, wherein the magnetic material is selected from the group consisting of ferromagnetic materials, ferrimagnetic materials, and paramagnetic materials. Non-limiting examples of the ferromagnetic materials include iron, nickel, cobalt, gadolinium, terbium, dysprosium, and alloys thereof, such as iron-nickel alloys, iron-cobalt alloys, nickel-cobalt alloys, carbon steel, stainless steel (ferromagnetic grades), and rare-earth magnets, including neodymium-iron-boron (NdFeB) and samarium-cobalt (SmCo). Non-limiting examples of the ferrimagnetic materials include magnetite (Fe₃O₄), maghemite (γ-Fe₂O₃), ferrites such as barium ferrite, strontium ferrite, cobalt ferrite, manganese ferrite, zinc ferrite, and lithium ferrite. Non-limiting examples of the paramagnetic materials include aluminum, platinum, chromium, manganese, palladium, and certain rare-earth elements, including erbium, europium, and holmium. In some embodiments, the magnetic material may further include magnetically ordered nanomaterials, including but not limited to, iron oxide nanoparticles, superparamagnetic iron oxide nanoparticles (SPIONs), and composite materials comprising magnetic particles embedded in a polymer, ceramic, or metallic matrix. As used herein, magnetic materials are also magnetically responsive.

In some embodiments, a MACS-based method can sort 200-300 million MSCs within 3-4 hours using four LS (medium-size) columns configured as in FIG. 1, when used simultaneously. Accordingly, the number of columns used can be scaled up to produce higher numbers of isolated MSCs. The following protocol can provide optimized sequential isolation methods of N-cad+ hWJMSCs (positive selection) and N-cad− hWJMSCs (negative selection). These isolated N-cad+ cells can then be used for transplantation into osteoarthritic knee joints, or other joint spaces, with or without hydrogel.

In some embodiments, the following method can be performed to obtain the cells: 1. Obtain hWJMSCs; 2. Shake hWJMSCs cells to recover N-Cadherin surface marker after dissociating with TrypLE; 3. Filter hWJMSCs cells through 70 μm cell strainer; 4. Incubate hWJMSCs cells with dead cell removal microbeads; 5. Incubate hWJMSCs cells with a Biotinylated N-Cadherin antibody; 6. Wash off unbound Biotinylated N-Cadherin antibody; 7. Incubate hWJMSCs cells bound to Biotinylated N-Cadherin antibody with anti-biotin microbeads (e.g., magnetically responsive); 8. Wash cells (e.g., once or more); 9. Apply hWJMSCs cells bound to Biotinylated N-Cadherin antibody with anti-biotin microbeads onto LS column; 10. Collect flow-through unlabeled live cells; 11. Collect flow-through unlabeled N-Cadherin negative cells; and 12. Flush out and optionally count the magnetically labeled N-Cadherin positive cells. Additionally, in some embodiments, immunostaining can optionally be performed to confirm N-cad+ MSCs.

In another embodiment, the collection method can be performed as follows: providing the sample of cells having the N-cad+ cells and non-N-cad+ cells; dissociating the cells; shaking the dissociated cells; filtering the shaken cells; removing dead cells from live cells; applying the live cells to a separation column system capable of providing a magnetic field; collecting flow-through cells; incubating the cells remaining in the separation column system with a biotinylated N-cadherin antibody; wash off unbound antibody; incubating the cells with an anti-biotin substrate; washing cells to remove anti-biotin substrate that is not bound to the biotinylated N-cadherin antibody; removing cells not bound to the biotinylated N-cadherin antibody (e.g., N-cad− cells); and collecting N-cad+ cells bound to the biotinylated N-cadherin antibody.

In another embodiment, the cell collection method can include: providing the sample of cells having the N-cad+ cells and non-N-cad+ cells; dissociating the cells; shaking the dissociated cells; filtering the shaken cells; removing dead cells from live cells; incubating the cells with a biotinylated N-cadherin antibody; washing off unbound antibody; incubating the cells with an anti-biotin substrate; washing cells to remove anti-biotin substrate that is not bound to the biotinylated N-cadherin antibody; removing cells not bound to the biotinylated N-cadherin antibody (e.g., N-cad− cells); and collecting N-cad+ cells bound to the biotinylated N-cadherin antibody.

In some embodiments, the present disclosure provides a method of sorting for N-cadherin positive (N-cad+) cells. This method may include providing a sample of cells having N-cad+ cells mixed with non-N-cad+ cells. The cells may be incubated with a N-cadherin antibody having a coupling region (e.g., biotinylated N-cadherin antibody). The cells may be incubated with a coupling substrate (e.g., anti-biotin substrate) that binds with the coupling region to form a coupling region and coupling substrate connection. Cells not bound to the N-cadherin antibody having a coupling region (e.g., biotinylated N-cadherin antibody) may be removed. The N-cad+ cells bound to the N-cadherin antibody having a coupling region (e.g., biotinylated N-cadherin antibody) may be collected. In some aspects, the a coupling region and coupling substrate coupling are any coupling pair of entities or moieties that couple with each other.

In certain embodiments, the composition comprises a pair of entities configured to bind or couple to each other, thereby linking two different moieties. The pair of entities may include, but is not limited to, biotin and streptavidin, biotin and avidin, biotin and neutravidin, or a biotinylated antibody and an anti-biotin antibody or an anti-biotin-coated substrate. Additional exemplary binding pairs include digoxigenin and anti-digoxigenin antibody, fluorescein and anti-fluorescein antibody, dinitrophenol (DNP) and anti-DNP antibody, HA-tag and anti-HA antibody, FLAG-tag and anti-FLAG antibody, myc-tag and anti-myc antibody, and His-tag and nickel-nitrilotriacetic acid (Ni-NTA). Other examples include glutathione and glutathione S-transferase (GST), an antigen and a cognate antibody, an oligonucleotide and its complementary oligonucleotide, DNA and a DNA-binding protein, RNA and an RNA-binding protein, and an aptamer and its cognate ligand.

Further examples include receptor-ligand pairs, enzyme and substrate analogs or inhibitors, peptide and major histocompatibility complex (MHC), and lectin-carbohydrate interactions. Binding pairs may also include streptamer and Strep-Tactin, as well as avidin analog systems such as desthiobiotin or iminobiotin. In certain embodiments, the binding pair comprises reactive chemical moieties such as azide and alkyne, strained alkyne and tetrazine, thiol and maleimide, thiol and haloacetamide, amine and N-hydroxysuccinimide (NHS) ester, aldehyde and hydrazide or aminooxy group, isothiocyanate and amine, epoxide and nucleophile, carbodiimide and carboxylic acid, or boronic acid and diol.

Photoreactive and electrostatic binding systems may also be employed, such as photo-cleavable groups and photoreactive groups, or polycation and polyanion interactions. Metal-mediated binding may include metal ions and their chelating groups. Additional protein-protein interaction pairs may include PDZ domains and PDZ-binding motifs, SH3 domains and proline-rich motifs, leucine zipper peptides, and coiled-coil peptide pairs. In certain embodiments, engineered or recombinant pairs may be used, such as SpyTag and SpyCatcher, SnoopTag and SnoopCatcher, HaloTag and HaloTag ligand, SNAP-tag and benzylguanine derivatives, CLIP-tag and benzylcytosine derivatives, or split enzyme fragments such as split green fluorescent protein (GFP) or split β-lactamase. Other inducible interaction systems may include FKBP and FRB in the presence of rapamycin. Multivalent or avidity-enhanced scaffold systems may also be used. Each of the foregoing binding pairs may include any analog, derivative, fusion, mimic, engineered variant, functional equivalent, or chemically modified form thereof.

In some embodiments, the above described MACS-based methods can sort 200-300 million MSCs within 3 hours for IA injections. In the present disclosure, the MACS-based sorting methods have been developed for isolating live N-cad+ and N-cad− hWJMSCs to meet the needs for IA cell therapy in animal studies and human clinical trials.

### Methods for Modifying Hydrogels for Joint Space Delivery

In some embodiments, modified methods of hydrogel preparation can be performed to obtain desired viscosity or other parameters to meet the need for IA cell delivery into joints, such as knee joints. In some aspects, the modification is to dilute the hydrogel and/or reduce the viscosity thereof. For example, in some embodiments, the modified hydrogel can pass through a 27 G needle.

Accordingly, one aspect of the present invention relates to medical devices and systems comprising needles for injection, aspiration, or fluid transfer, wherein the needle has a gauge size (G) selected from the group consisting of 25 gauge, 26 gauge, 27 gauge, 28 gauge, 29 gauge, and 30 gauge. The needle may be configured as a hypodermic needle, intravenous needle, intramuscular needle, subcutaneous needle, microcannula, or other medical needle, and may be manufactured from stainless steel, titanium, polymer, or composite materials. The needle may further include a beveled tip, a blunt tip, or a tapered tip, and may be coated with a lubricating material, such as silicone, polytetrafluoroethylene (PTFE), or other biocompatible coatings. The needle may be attached to a syringe, catheter, infusion set, or other medical device, and may be provided in a sterile, single-use, or reusable form.

### Methods for Delivering Live N-cad+ and/or N-cad− WJMSCs into Joint Space

In some embodiments, methods for delivering N-cad+/N-cad− WJMSCs and other MSCs into joints (e.g., knee joints) can be performed as described herein. In some aspects, treatments with these cells, such as N-cad+ hWJMSCs, can be delivered into knee joints through intraarticular (IA) injection. For example, ultrasound Doppler-guided technique can be used to ensure the anatomic location of cell delivery. In some aspects, the cells can be delivered by IA injection into knee joints without ultrasound. In some aspects, other joints can receive the cells for treatment of joint pain with or without ultrasound or other visual detection technique.

In some embodiments, the N-cad+ and/or N-cad− WJMSCs can be delivered into the joint without leakage in studies for N-cad+ efficacy or other assay. In some aspects, the cells are N-cad+ cells when used for therapeutic treatments. In some aspects, the delivery omits N-cad− cells when used for therapeutic treatments.

In some embodiments, a two-angle puncture/insertion technique (e.g., two or more punctures) can be used for the injection, using a 30G1 needle for MSCs without hydrogel and a 27 G1/2 needle for MSCs encapsuled with hydrogel. This technique can prevent the IA injection of MSCs from having needle-hole leakage and ensure injected materials are diffused into the entire synovial cavities of both the femorotibial joint and the patellofemoral joint. It can be desirable to obtain 100% success, which was achieved for rat knee IA injections by experienced individuals. In some embodiments, the different injections can be at different angles with respect to each other or with respect to normal. In some embodiments, the two-angle method can make a first injection into one of the synovial cavities of the femorotibial and/or patellofemoral joints, and the second injection can be into the other.

### Methods of Treatment with N-Cad+ WJMSCs

The IA injection of WJMSCs with or without hydrogel delivery can be used for therapeutic effects. However, WJMSCs in hydrogel may be better than the WJMSCs alone for OA treatment (e.g., posttraumatic knee OA). The use of N-cad+ cells can provide an improvement in the treatment of OA. In some aspects of the present disclosure, WJMSCs with IA hydrogel delivery may have better therapeutic effects than WJMSCs alone on posttraumatic knee OA and articular cartilage repair.

### EXAMPLES

The following examples are provided to further illustrate the compositions and methods of the present disclosure. These examples are illustrative only and are not intended to limit the scope of the disclosure in any way.

### Example 1—Isolating Live N-cad+ and N-cad− hWJMSCs

A sequential procedure was used for isolating live N-cad+ and N-cad− hWJMSCs. Mixed hWJMSCs containing both N-cad+ and N-cad− hWJMSCs were used to show the selective sorting. Dead cells produced during the cell culture and expansion were removed using a Dead Cell Removal Kit. Cells were then incubated with a biotinylated N-cadherin antibody at 4° C. for 10 min. After washing off unbound primary antibody, cells were incubated with 20 μL of Anti-Biotin MicroBeads at 4° C. for 15 min. After washing off unbound microbeads, cells were applied onto a rinsed MACS LS column. After washing the column three times with 3 mL of MACS buffer, magnetically labeled cells were immediately flushed out from the column with 5 mL of MACS buffer. After Trypan-Blue exclusion staining, live N-cad+ and N-cad− cell numbers were counted using hemocytometry under an inverted microscope and the total number of live N-cad+ and N-cad− hWJMSCs was calculated and recorded.

### Example 2—Modifying Hydrogels for Joint Space Delivery

To perform cell engraftment and survival after MSC transplantation, three different hydrogels were used as vehicles for cell delivery: 1. HyStem-C Hydrogel Kit (Thiol-Modified Hyaluronan and Gelatin Hydrogel Kit) purchased from Advanced BioMatrix (ABM), San Diego, CA; 2. PepgelpGmatrixCDX/PDX, purchased from Pepgel, LLC, Manhattan, KS; and 3. N-cad (Ac-HAVD) peptide hydrogel (published information, Proc Natl Acad Sci USA, 2013. 110:10117-22); designed by L Li (SIMR) and synthesized by Pepgel, LLC, and, named: pGmatrix-HAVD CDX.

These hydrogels made with manufacturer recommended methods were too thick/stiff for IA injection. The hydrogel formation protocols can be modified from the manufacturer recommended methods of hydrogel preparation to meet the need for IA cell delivery. The modification can be implemented to decrease the concentration of the hydrogel, e.g., by dilution. The viscosity can thereby be reduced. The hydrogels can be tailored to pass through a 27 G needle.

To find the right hydrogel concentration for injection using a 27 G needle, a mixture of 3 million cells was tested with the hydrogel at the concentration of 50%, 60% and 70%, which provides a suitable range. It was also found that the formula having about 60% hydrogel gave the optimal injection condition. Nonetheless, the hydrogel thickness or viscosity can be modulated by dilution as needed or desired for injection or other implantation.

For example, a standard formula (70% hydrogel) that can pass a 25 G needle was prepared by: (1) Resuspend cells in medium to the density of 3 million per 75 μl; (2) Add 70 μL of Glycosil and 70 μl Gelin-S into a new microtube, pipette to mix; (3) Add 75 μL of the cells (3 million) to the Glycosil+Gelin-S mix, pipette to mix; (4) Add 35 μL of Extralink to the tube, pipette to mix; and (5) Save the cell-gel mixture on ice for injection.

In another example, the modified hydrogel preparation can include a 60% hydrogel formula that can easily pass a 27 G needle by: (1) Resuspend cells in medium to the density of 3 million per 100 μL; (2) Add 60 μL of Glycosil and 60 μL Gelin-S into a new microtube, pipette to mix; (3) Add 100 μL of the cells (3 million) to the Glycosil+Gelin-S mix, pipette to mix; (4) Add 30 μL of Extralink to the tube, pipette to mix; and (5) Save the cell-gel mixture on ice for injection.

Similarly, a PGmatrix CDX gel was used at the concentration of 40%, 50% and 60% with the mixture of 3 million cells and found that 3 million cells in 50% hydrogel formula gave the optimal injection condition. Standard formula (50% hydrogel) that can pass a 25 G needle can be prepared by: (1) Resuspend 3 million cells in 112.5 μL medium; (2) Add 12.5 μL of Solution A: PGmatrrix trigger to the cell suspension, gently mix well; (3) Add 125 μL of Solution B: PGmatrrix matrix solution to the cell suspension, pipette to mix without introducing air bubbles; and (4) Save the cell-gel mixture on ice for injection.

In a further example, a modified hydrogel preparation can be a 40% hydrogel formula that can easily pass a 27 G needle, which can be prepared by: (1) Resuspend 3 million cells in 140 μL medium; (2) Add 10 μL of Solution A: PGmatrrix trigger to the cell suspension, gently mix well; (3) Add 100 μL of Solution B: PGmatrrix matrix solution to the cell suspension, pipette to mix without introducing air bubbles; and (4) Save the cell-gel mixture on ice for injection.

Accordingly, other variations and dilutions of the hydrogel can be achieved.

### Example 3—Delivering Live N-cad+ and/or N-cad− hWJMSCs into Joint Space

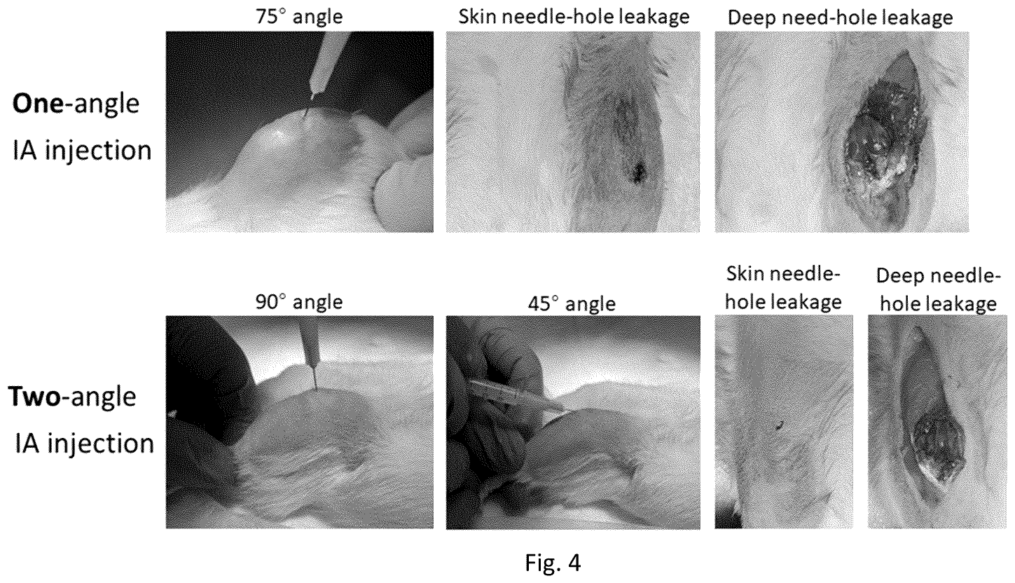

Preliminary experiments revealed a high incidence of needle-hole leakage and insufficient IA diffusion of transplanted cells when using the prior published methods of IA injections. In the present disclosure, a two-angle insertion (e.g., puncture) technique was performed to inhibit or reduce leakage from the injection hole. This two-angle puncture/insertion technique can prevent the IA injection of MSCs from needle-hole leakage and ensure injected materials are diffused into the entire synovial cavities of both the femorotibial joint and the patellofemoral joint. This two-angle puncture/insertion technique demonstrated 100% success for rat knee IA injections (FIG. 4). Therefore, the two-angle puncture/insertion technique can also be applied to humans and other joints. Moreover, a multi-angle puncture/insertion technique with two or more injections can be performed, where the total amount of cell delivery can be split between the injections.

In order to have a suitable test model, a medial meniscotibial ligament transection (MMLT) was used to generate destabilization of the medial meniscus (DMM) to induce a knee OA model. These MMLT animals were treated with a single intra-articular (IA) injection of specific MSCs into the knee joint bilaterally in 9-12 weeks old normal rats. Various types of MSCs or PBS (placebo/negative control) were transplanted into rat osteoarthritic knee joints induced by DMM via IA injection at 4 weeks after the DMM surgery.

Various types of MSCs or PBS were delivered via IA injection into rat osteoarthritic knee joints induced by surgical destabilization of the medial meniscus (DMM) at 4 weeks after the knee injury procedure. Animals were treated with different types of stem cells or PBS as follows: 1) human adipose-derived MSCs (Ad) as a comparator, 2) human bone marrow-derived MSCs (BM) as a comparator, 3) mixed human WJMSCs containing both N-cad+ and N-cad− MSCs (MWJ), 4) human N-Cad+ bone marrow MSCs (Ncd+BM), 5) human N-Cad+ WJMSCs (Ncd+WJ), 6) human N-cad−bone marrow MSCs (Ncd−BM), 7) human N-cad− WJMSCs (Ncd−WJ), and 8) phosphate buffered saline (PBS) as a negative control or placebo. Histopathologic analysis of the rat knee joints from 8 different treatment groups harvested at 4 weeks after IA treatment (8 weeks after surgical DMM knee injury) showed osteoarthritic changes with various severity scores which were assessed by 3 independent observers using an internationally recognized scoring system in all 8 groups. For example, 1.5×10⁶ live cells per joint were introduced into the knees unilaterally or bilaterally at 4 weeks post-surgery, at which time early knee OA is usually observed in mice and rats.

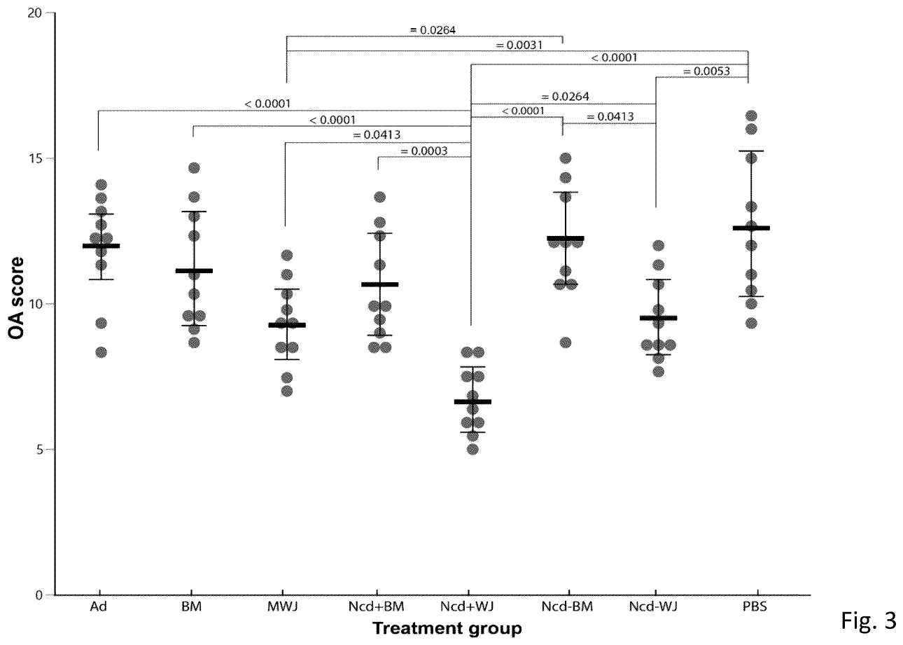

Statistical analysis of therapeutic effects of various treatments on rat knee OA was performed, and the results were shown in FIG. 3. One-way ANOVA analysis revealed a significant overall difference in OA score/severity among the 8 groups (p<0.0001). Post hoc Tukey tests (using JMP Pro Statistical Software, Version 16) demonstrated that N-cad+ hWJMSC group had the least severe OA changes (best therapeutic effect, p<0.0001 vs. PBS) among the 8 treatment groups, while other types of hWJMSCs less effectively attenuated OA progression (MWJ: p=0.0031 vs. PBS, Ncd−WJ: p=0.0053 vs. PBS).

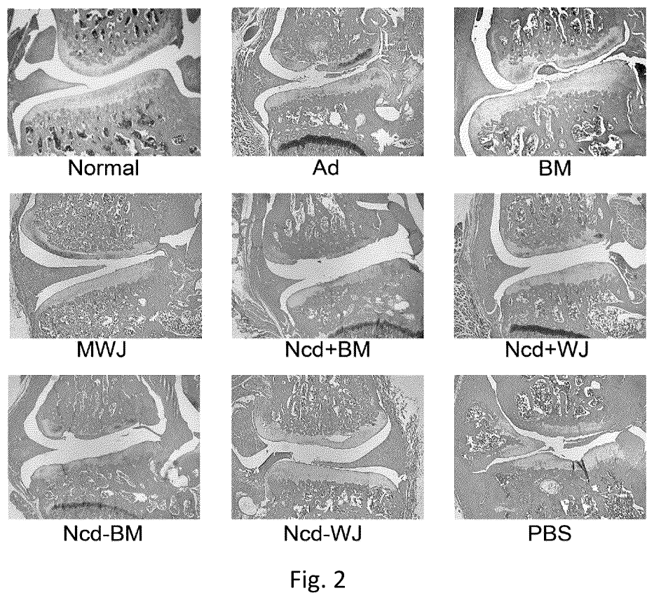

The data showed that the N-cad+ MSCs have better therapeutic effects on rat knee OA than other types of MSCs that are currently used for preclinical animal studies and human clinical trials. This result indicates the N-cad+ hWJMSC-based products can be used for treatments of OA in humans. Histopathologic analysis of the knee joints from the 8 different treatment groups harvested at 4 weeks after treatment (8 weeks after surgical knee injury) demonstrated osteoarthritic changes with various severities in all 8 groups. The data suggest that none of the treatments can completely reverse the progression of DMM-induced posttraumatic knee OA in rats (see, FIG. 2).

FIG. 2 includes representative photomicrographs that show various levels of OA severity in the knee joints receiving different IA treatments at 4 weeks after the DMM surgery, except for the unoperated normal control knee. The staining was safranin-O and fast-green staining, counterstained with hematoxylin. Cartilage cells and matrices were stained in red or orange. Normal=unoperated normal control knee; Ad=treated with human adipose-derived MSCs; BM=human bone marrow-derived MSCs; MWJ=mixed human WJMSCs containing both N-cad+ and N-cad− WJMSCs; Ncd+BM=human N-Cad+ bone marrow MSCs; Ncd+WJ=human N-Cad+ WJMSCs; Ncd−BM=human N-cad-bone marrow MSCs; Ncd−WJ=human N-cad− WJMSCs; PBS=phosphate buffered saline. N=10 (5 females+5 males) per treatment group per time point. Semiquantitative OA grading suggested that N-cad+ hWJMSC group had least OA severity (best therapeutic effect) among the 8 treatment groups at 4 weeks after treatment. However, none of the treatments could completely reverse OA progression.

In the present disclosure, a protocol for histopathologic OA grading is provided. The protocol measured the histopathologic OA severity of tissue sections from the rat knee joints using a semiquantitative OA grading system published by the Osteoarthritis Research Society International (OARSI). Representative midcoronal sections of the knee joints were used for scoring in this study because previous studies have confirmed that osteoarthritic changes are more severe in the weight-bearing area (mid-portion) of rodent knee joints and that a single midcoronal section can consistently define OA severity. OA severity was scored by three independent observers (scorers) who have knowledge of the knee structure, OA histopathology, and the published grading criteria for rat knee OA. All three observers were blinded to the information on animal groups and timepoints to ensure unbiased assessments. Since DMM-induced OA changes mainly affect the medial compartment of the knee joint, averaged medial compartment OA scores from three observers were utilized for statistical analyses.

Comparative analysis of therapeutic efficacy was performed for different treatments for rat knee OA. Statistical analysis of therapeutic effects of various MSC treatments on posttraumatic knee OA demonstrated that while all three WJMSC-based treatments significantly attenuated OA progression, N-cad+ hWJMSC group showed the least severe OA changes among the 8 treatment groups at 4 weeks after treatment. That is, the N-cad+ hWJMSC group provided the best treatment for the OA model. The comparisons among 8 groups with statistically significant differences are presented in FIG. 3. Ordered differences in OA score among all groups are presented in Table 1.

FIG. 3 includes a scatter plot that shows the distribution of OA score points for each of the 8 treatment groups. The short horizontal lines within each group represent the mean (thicker line) and standard deviation error bars (thin lines), respectively. The numerical numbers above the grouped score points represent p-values of statistical comparisons (Tukey tests) among 8 groups with a statistically significant difference.

TABLE 1
Ordered differences report of One-way ANOVA and Tukey HSD analysis

| | | | Std Err | Lower | Upper | |
|---|---|---|---|---|---|---|
| Treatment group | | Diff | Diff | CL | CL | p-Value |
| PBS | Ncd + WJ | 5.966667 | 0.824696 | 3.39212 | 8.541216 | <.0001 |
| Ncd − BM | Ncd + WJ | 5.4 | 0.824696 | 2.82545 | 7.974549 | <.0001 |
| Ad | Ncd + WJ | 5.133333 | 0.824696 | 2.55878 | 7.707883 | <.0001 |
| BM | Ncd + WJ | 4.5 | 0.824696 | 1.92545 | 7.074549 | <.0001 |
| Ncd + BM | Ncd + WJ | 3.866667 | 0.824696 | 1.29212 | 6.441216 | 0.0003 |
| PBS | MWJ | 3.333333 | 0.824696 | 0.75878 | 5.907883 | 0.0031 |
| PBS | Ncd − WJ | 3.2 | 0.824696 | 0.62545 | 5.774549 | 0.0053 |
| Ncd − BM | MWJ | 2.766667 | 0.824696 | 0.19212 | 5.341216 | 0.0264 |
| Ncd − WJ | Ncd + WJ | 2.766667 | 0.824696 | 0.19212 | 5.341216 | 0.0264 |
| Ncd − BM | Ncd − WJ | 2.633333 | 0.824696 | 0.05878 | 5.207883 | 0.0413 |
| MWJ | Ncd + WJ | 2.633333 | 0.824696 | 0.05878 | 5.207883 | 0.0413 |
| Ad | MWJ | 2.5 | 0.824696 | −0.07455 | 5.074549 | 0.0632 |
| Ad | Ncd − WJ | 2.366667 | 0.824696 | −0.20788 | 4.941216 | 0.0943 |
| PBS | Ncd + BM | 2.1 | 0.824696 | −0.47455 | 4.674549 | 0.1933 |
| BM | MWJ | 1.866667 | 0.824696 | −0.70788 | 4.441216 | 0.3279 |
| BM | Ncd − WJ | 1.733333 | 0.824696 | −0.84122 | 4.307883 | 0.4237 |
| Ncd − BM | Ncd + BM | 1.533333 | 0.824696 | −1.04122 | 4.107883 | 0.5827 |
| PBS | BM | 1.466667 | 0.824696 | −1.10788 | 4.041216 | 0.6364 |
| Ad | Ncd + BM | 1.266667 | 0.824696 | −1.30788 | 3.841216 | 0.7855 |
| Ncd + BM | MWJ | 1.233333 | 0.824696 | −1.34122 | 3.807883 | 0.8073 |
| Ncd + BM | Ncd − WJ | 1.1 | 0.824696 | −1.47455 | 3.674549 | 0.8828 |
| Ncd − BM | BM | 0.9 | 0.824696 | −1.67455 | 3.474549 | 0.9567 |
| PBS | Ad | 0.833333 | 0.824696 | −1.74122 | 3.407883 | 0.9714 |
| Ad | BM | 0.633333 | 0.824696 | −1.94122 | 3.207883 | 0.9942 |
| BM | Ncd + BM | 0.633333 | 0.824696 | −1.94122 | 3.207883 | 0.9942 |
| PBS | Ncd − BM | 0.566667 | 0.824696 | −2.00788 | 3.141216 | 0.9971 |
| Ncd − BM | Ad | 0.266667 | 0.824696 | −2.30788 | 2.841216 | 1 |
| Ncd − WJ | MWJ | 0.133333 | 0.824696 | −2.44122 | 2.707883 | 1 |

There appears to be no differences between sexes. No significant differences in OA scores were found between male and female animals in any of the treatment groups at 4 weeks after IA treatment. However, OA sores were found higher in male animals than female animals at 12 weeks after IA treatment with various types of MSCs. According to the data, N-cad+ hWJMSCs have better therapeutic effects on knee OA in rats. Accordingly, the N-cad+ hWJMSCs can be used in intra-articular (IA) injection of hWJMSCs with or without hydrogel. The N-cad+ hWJMSCs can provide better therapeutic effects than other types of human MSCs (BM, Adipose). The data showed better results for N-cad+ hWJMSCs for treatment of posttraumatic knee OA in normal immune competent rats at 4 and 12 weeks after IA treatment. Thus, the N-cad+ hWJMSCs can also be used for humans and in other joints to treat OA.

### Example 4—Treatment with N-Cad+ hWJMSCs

Preliminary experiments revealed a high incidence of needle-hole leakage with insufficient IA diffusion when using the prior published methods of IA injection. In this study, a two-angle injection technique was developed to prevent the needle-hole leakage of MSCs to ensure injected materials being diffused into the entire synovial cavities of both the femorotibial joint and the patellofemoral joint.

FIG. 4 illustrates a one-angle IA injection at a 75° angle, which resulted in skin needle-hole leakage and deep needle-hole leakage. The two-angle IA injection at 90° angle and 45° angle (in either order or sequence), showed minimal to no skin needle-hole leakage or deep needle-hole leakage. Accordingly, two or more sequential injections at different angles can be used to provide the N-cad+ hWJMSCs.

While 90° angle and 45° angle were exemplified, various different degrees and degree combinations can be used. For example, the two different angles can be at least 10° different, 20° different, 30° different, or 40° different or more. Also, at least one angle can be 90° or normal, or between 80° and 100° or between 70° and 120° or between 60° and 130°.

In FIG. 4, photographs showed the visional difference in needle-hole leakage and intra-articular (IA) diffusion between one-direction and two-direction IA injection methods, which were performed on freshly euthanized rat cadavers. The amount of needle-hole leakage of Alcian blue dye containing MSCs was substantially less for the two-direction method than the one-direction method, while the amount of dye diffusion in the femoral groove (patellofemoral joint) was substantially more for the two-direction method than the one-direction method.

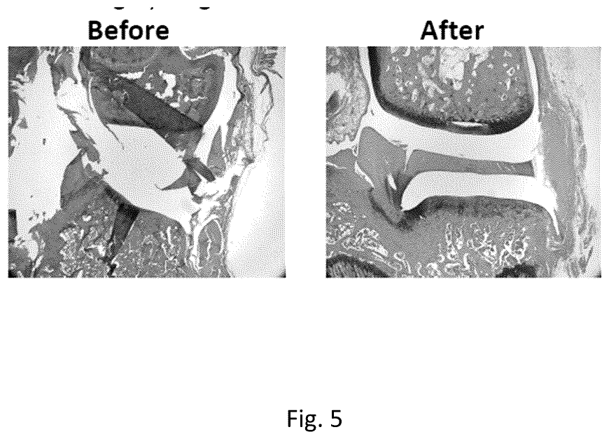

To overcome the technical challenge that tissue sections from rat knee joints injected with hydrogel alone or hydrogel-encapsulated MSCs had poor adhesion to glass slides and were easy to peel off during staining, the routine histological processing methods were modified as follows: 1) increasing paraffin infiltration time from the routine 65° C., 2 hours (1 hour×2 changes under vacuum) to 65° C., 4 hours (2 hours×2 changes under vacuum); 2) replacing the routine safranin-O/Fast Green staining method with a modified method by adding a section-drying procedure (56° C. for 60 minutes) after hematoxylin stain and before Safranin-O stain to improve section adhesion to the glass slides. This modification essentially resolved the tissue adhesion issue and substantially improved the quality of staining (FIG. 5). FIG. 5. includes photomicrographs showing the difference in tissue adhesion and staining quality before and after the modification of the method for processing tissue sections and staining procedure.

In some instances, N-cad+ hWJMSCs can live longer in vivo after IA injection due to their lower immunogenicity and higher biocompatibility, thereby exerting better therapeutic effects than other types of human MSCs for treatment of knee OA, as evidenced in normal rats. Joint tissues sections with and without hydrogel encapsulation can be applied to glass slides for histology. Conducting qPCR gene expression analysis can be used to measure the expression levels of pro-inflammatory and anti-inflammatory cytokines and immune cell markers in all treatment groups.

### Example 5—Gene Expression Changes

The same knee OA model was induced in rats by surgical destabilization of the medial meniscus (DMM). Intra-articular treatment (e.g., described herein) was performed generally by: 4 weeks after surgical DMM knee injury, animals were treated through intra-articular injection (IA) of the following agents: 1) human adipose-derived MSCs (Ad) as a comparator, 2) human bone marrow-derived MSCs (BM) as a comparator, 3) human N-cad−bone marrow MSCs (Ncd−BM), 4) human N-Cad+ bone marrow MSCs (Ncd+BM), 5) mixed human umbilical cord (Wharton's jelly/WJ) derived MSCs (WJMSCs) containing both N-cad+ and N-cad− MSCs (MWJ), 6) human N-Cad+ WJMSCs (Ncd+WJ), 7) human N-cad− WJMSCs (Ncd−WJ), and 8) phosphate buffered saline (PBS) as a negative control or placebo.

Gene expression analysis was performed after treatment with the N-cad+ hWJMSCs and control groups. Quantitative real-time reverse transcription polymerase chain reaction (qRT-PCR) was performed to analyze the expression levels of genes of interest in articular cartilage (AC/cartilage) and synovium of the knee OA joints treated with different agents as described above. Knee joint cartilage and synovium (containing a thin layer of joint capsular tissue due to technical difficulties in obtaining pure synovium) were freshly collected under a microscope for RNA isolation and qPCR analysis. Gene expression levels from the joint tissue samples were quantified using 2-ΔΔCt methods. Specific primers used in this experiment are for: Acan (NM_022190); Adamts5 (AY382879); CD163 (NM_00107887.1); Col2a1 (NM_001414896); Il-10 (NM_012854); Il-1β (NM_031512); FoxP3 (NM_001108250.1); Prg4 (NM_001105962); Tnf-α(NM_012675); and Gapdh (AF106860.2). Any suitable primer set of forward and reverse primers can be used.

It was found that there were gene expression changes in the articular cartilage in the various treatment groups. The gene analysis was conducted on the Ad, BM, MWJ, Ncd+WJ, Ncd−WJ, and PBS (control) groups because the histopathological analysis of OA severity showed no significant difference among the BM, Ncd−BM, and Ncd+BM groups. The following genes of interest known to be involved in the development of osteoarthritic cartilage lesions were analyzed: 1) Acan (encoding aggrecan, a structural protein in the cartilage); Col2a1 (encoding type-II collagen, a structural protein in the cartilage and a marker of the chondrocyte); Il-10 (encoding interleukin-10, a key anti-inflammatory cytokine protecting the body from excessive tissue damage); Il-1b (encoding interleukin-1β, a pro-proinflammatory cytokine); Tnf-α (encoding tumor necrosis factor-α, a key player in inflammation and cell death); Adamts5 (encoding a disintegrin and metalloproteinase with thrombospondin motifs-5), an enzyme cleaves aggrecan and contributes to cartilage degradation).

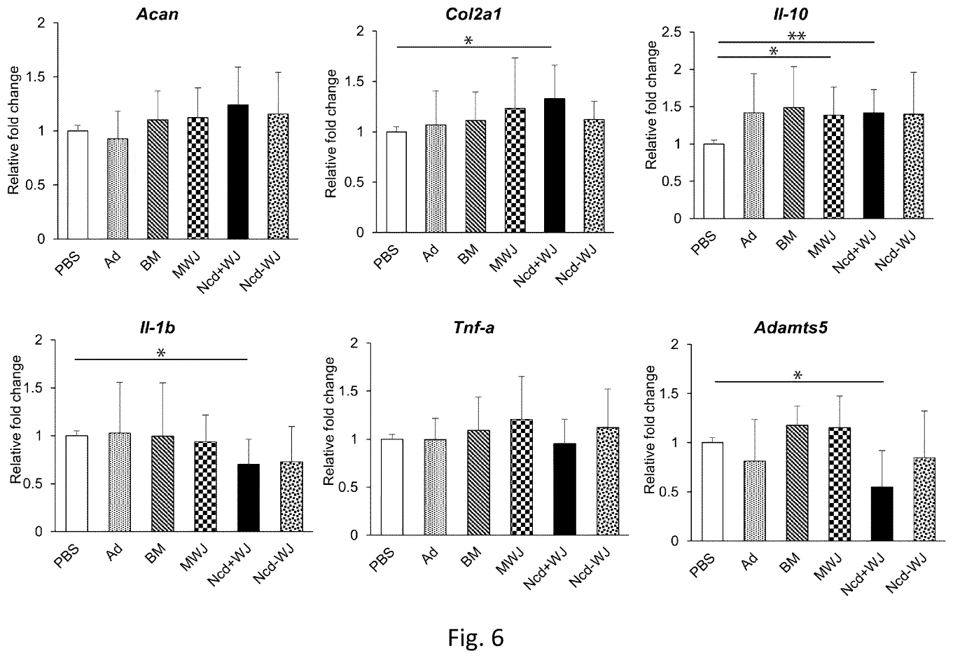

The gene expression levels in cartilage at 4 weeks after IA treatment (8 weeks after DMM surgery) are presented in FIG. 6. FIG. 6 shows the relative fold change against PBS, AD, BM, MWJ, Ncd+WJ and Ncd−WJ cell groups for Can, Col2a1, Il-10, Il-1b (e.g., Il-1β), Tnf-α (e.g., Tnf-α), and Adamts5. The data is from a qPCR gene expression analysis with Student's t-tests (unpaired, two-tailed) showing differential expression of anabolic and catabolic genes in knee joint cartilage harvested from at 4 weeks following IA treatment. The expression level of each age-matched PBS group has been normalized to “1.0”. *p<0.05, **p<0.01. N=6 rats per time point/treatment group.

It was found that there were gene expression changes in synovium of various treatment groups. For the reason described above, the study focused the gene analysis on the Ad, BM, MWJ, Ncd+WJ, Ncd−WJ, and PBS (control) groups. The following genes of interest known to be involved in the development of osteoarthritic synovitis were analyzed: Prg4 (encoding lubricin, a large mucin-like proteoglycan that plays a crucial role in boundary lubrication in joints and other tissues); CD163 (encoding hemoglobin scavenger receptor (HbSR), which is highly expressed in M2 macrophages associated with anti-inflammatory responses and tissue repair; Il-1β (encoding interleukin-1β, a pro-proinflammatory cytokine); FoxP3 (encoding FOX3 protein, a transcription factor that plays a crucial role in the development and function of regulatory T cells (Tregs); Tnf-α (encoding tumor necrosis factor-α, a key player in inflammation and cell death); Adamts5 (encoding a disintegrin and metalloproteinase with thrombospondin motifs-5, an enzyme cleaves aggrecan and contributes to cartilage degradation).

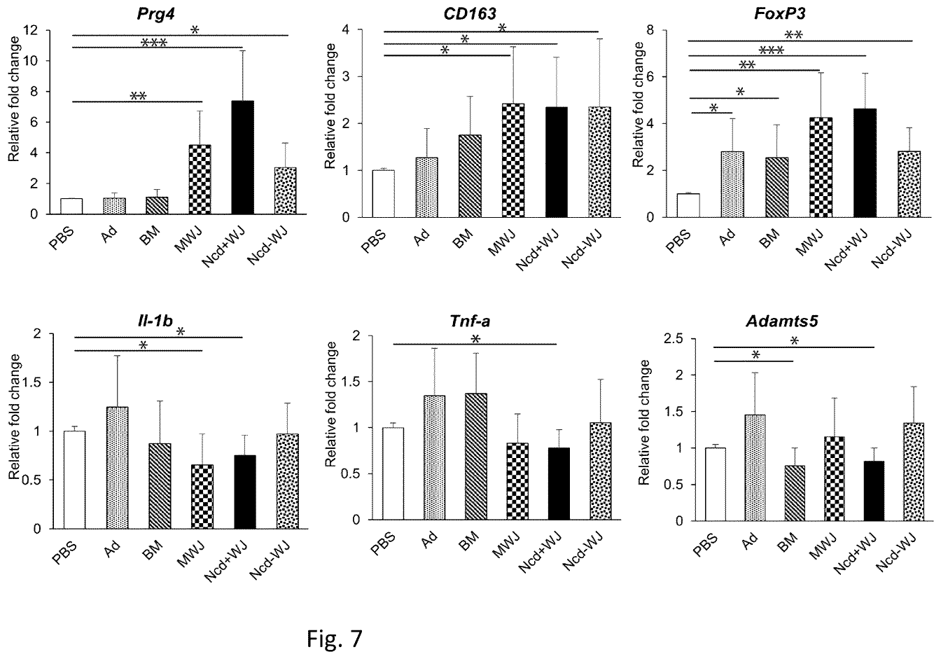

The gene expression levels in synovium at 4 weeks after IA treatment (8 weeks after DMM surgery) are presented in FIG. 7. FIG. 7 includes the same cell groups with the relative fold change for Prg4, CD163, FoxP3, Il-1β, Tnf-α, and Adamts5. The qPCR gene expression analysis was performed with Student's t-tests (unpaired, two-tailed) showing differential expression of anabolic and catabolic genes in knee joint synovium harvested from at 4 weeks following IA treatment. The expression level of each PBS group was normalized to “1.0”. *p<0.05, **p<0.01, ***p<0.001. N=6 rats per time point/treatment group.

Accordingly, all the analyzed MSCs (e.g., Ad, BM, MWJ, Ncd+WJ, and Ncd−WJ) significantly upregulated FoxP3 expression and all 3 types of umbilical cord (WJ)-derived MSCs (MWJ, Ncd+WJ, and Ncd−WJ) significantly upregulated Prg4 expression in the synovium of rat knee joints with OA. These results suggest that all 5 types of analyzed MScs may moderate the function of regulatory T cells and all WJ-derived MSCs can promote the production of lubricin that lubricates the knee joint.

Among the 5 different treatments analyzed here, the N-cad+ hWJMSC (Ncd+WJ) treatment promoted the expression of more anabolic or anti-inflammatory genes and suppressed the expression of more catabolic genes in cartilage and synovium than any other types of tested MSCs. This is consistent with the histopathological analysis showing that the therapeutic effect of the N-cad+ hWJMSC was superior to other types of MSCs for treatment of posttraumatic knee OA in rats.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### REFERENCES

CDC: Center for Disease Control and Prevention. Osteoarthritis (OA). Jan. 26, 2024. (CDC) CfDCaP. Current prevalence of osteoarthritis. 2024 [updated Jan. 26, 2024]; Available from: cdc.gov/arthritis/osteoarthritis/index.html Lawrence, R.C., et al., Estimates of the prevalence of arthritis and other rheumatic conditions in the United States. Part II. Arthritis Rheum, 2008. 58(1): p. 26-35.
Le Graverand-Gastineau, M.P., Disease modifying osteoarthritis drugs: facing development challenges and choosing molecular targets. Curr Drug Targets, 2010. 11(5): p. 528-35.
Hellio le Graverand, M.P., et al., A 2-year randomised, double-blind, placebo-controlled, multicentre study of oral selective iNOS inhibitor, cindunistat (SD-6010), in patients with symptomatic osteoarthritis of the knee. Ann Rheum Dis, 2013. 72(2): p. 187-95.
Caldwell, K.L. and J. Wang, Cell-based articular cartilage repair: the link between development and regeneration. Osteoarthritis Cartilage, 2015. 23(3): p. 351-62.
Jevotovsky, D.S., et al., Osteoarthritis and stem cell therapy in humans: a systematic review. Osteoarthritis Cartilage, 2018. 26(6): p. 711-729.
Bian, L., et al., Hydrogels that mimic developmentally relevant matrix and N-cadherin interactions enhance MSC chondrogenesis. Proc Natl Acad Sci U S A, 2013. 110(25): p. 10117-22.
Zhao, M., et al., N-Cadherin-Expressing Bone and Marrow Stromal Progenitor Cells Maintain Reserve Hematopoietic Stem Cells. Cell Rep 2019. 26(3): p. 652-669.e6.
Cheng, J.H., et al. Comparison efficacy of ESWT and Wharton's jelly mesenchymal stem cell in early osteoarthritis of rat knee. Am J Transl Res 2019. 11:586-598.
Kanaya, A., et al. Intra-articular injection of mesenchymal stromal cells in partially torn anterior cruciate ligaments in a rat model. Arthroscopy 2007. 23:610-617.
Gerwin N, et al. The OARSI histopathology initiative-recommendations for histological assessments of osteoarthritis in the rat. Osteoarthritis Cartilage 2010; 18 Suppl 3: S24-34.
Sutermaster B.A. et al. Considerations for high-yield, high-throughput cell enrichment: fluorescence versus magnetic sorting. Scientific Report 2019; 9:227 |DOI: 10.1038/s41598-018-36698-1.
Kotlarz H, Gunnarsson CL, Fang H, Rizzo JA. Insurer and out-of-pocket costs of osteoarthritis in the US: evidence from national survey data. Arthritis Rheum 2009; 60:3546-3553.
Glasson SS, Chambers MG, Van Den Berg WB, Little CB. The OARSI histopathology initiative—recommendations for histological assessments of osteoarthritis in the mouse. Osteoarthritis Cartilage 2010; 18 Suppl 3: S17-23.
McNulty MA, Loeser RF, Davey C, Callahan MF, Ferguson CM, Carlson CS. A Comprehensive Histological Assessment of Osteoarthritis Lesions in Mice. Cartilage 2011; 2:354-363.
Armstrong AR, Carlson CS, Rendahl AK, Loeser RF. Optimization of histologic grading schemes in spontaneous and surgically-induced murine models of osteoarthritis. Osteoarthritis Cartilage 2021; 29:536-546.
(Livak KJ, Schmittgen TD. Analysis of relative gene expression data using real-time quantitative PCR and the 2(-Delta Delta C(T)) Method. Methods (San Diego, Calif). 2001 December; 25(4): 402-8.)
U.S. Pat. Nos. 8,263,403; 9,771,559; and U.S. Pat. No. 9,896,659.

1. A method of sorting for N-cadherin positive (N-cad+) cells comprising:
providing a sample of cells having N-cad+ cells and with non-N-cad+ cells;
incubating the cells with a N-cadherin antibody having a coupling region;
incubating the cells with a coupling substrate that binds with the coupling region;
removing cells not bound to the N-cadherin antibody; and
collecting the N-cad+ cells.

2. The method of claim 1, wherein the sample of cells includes Wharton's jelly mesenchymal stem cells (WJMSCs) and the N-cad+ cells are N-cad+ WJMSCs.

3. The method of claim 1, further comprising:
dissociating the cells in the sample from each other before incubation.

4. The method of claim 3, further comprising:
shaking the dissociated cells sufficiently to present N-cadherin on cell surfaces.

5. The method of claim 4, further comprising:
filtering the cells after shaking.

6. The method of claim 5, further comprising:
removing dead cells from the filtered cells.

7. The method of claim 5, wherein the cells are filtered
with a 80-100 micron cell strainer, 60-80 micron cell strainer, or 30-60 micron cell strainer.

8. The method of claim 1, wherein the N-cadherin antibody is biotinylated and the coupling substrate is an anti-biotin substrate.

9. The method of claim 1, wherein the coupling substrate comprises microbeads.

10. The method of claim 1, wherein the coupling substrate comprises magnetically-responsive microbeads or magnetic microbeads.

11. The method of claim 10, further comprising applying a magnetic field to the cells after incubation with the coupling substrate.

12. The method of claim 10, further comprising placing the cells after incubation with the coupling substrate in a separation system having a magnetic field.

13. The method of claim 12, further comprising allowing cells not bound to the N-cadherin antibody to separate from the cells bound with the N-cadherin antibody attached to the microbeads.

14. The method of claim 13, further comprising collecting N-cad− cells not bound to the N-cadherin antibody, wherein the cells bound with the N-cadherin antibody attached to the microbeads are retained by the magnetic field.

15. The method of claim 14, further comprising:
removing the magnetic field from the cells bound with the N-cadherin antibody attached to the microbeads; and
collecting the cells bound with a N-cadherin antibody attached to the microbeads.

16. The method of claim 1, further comprising:
washing unbound N-cadherin antibody before further incubating the cells with the coupling substrate.

17. The method of claim 10, further comprising:
washing the cells after incubation with the coupling substrate.

18. The method of claim 12, after placing the cells in the separation system having the magnetic field, further comprising:
washing the cells before collecting any of the cells.

19. The method of claim 15, further comprising:
removing the microbeads from the collected cells.

20. A method of sorting for N-cadherin positive (N-cad+) cells comprising:
providing a sample of cells having N-cad+ cells and non-N-cad+ cells;
dissociating the cells in the sample;
shaking the dissociated cells;
filtering the shaken cells;
removing dead cells from the shaken cells to obtain live cells;
incubating the live cells with a N-cadherin antibody;
washing off unbound N-cadherin antibody;
further incubating the cells with a coupling substrate;
washing the cells to remove coupling substrate that is not bound to the N-cadherin antibody;
removing cells not bound to the N-cadherin antibody; and
collecting N-cad+ cells bound to the N-cadherin antibody.

21. The method of claim 20, wherein the cells not bound to the N-cadherin antibody are N-cad− cells.

22. A method of treating a joint disease in a subject, comprising:
providing N-cadherin positive (N-cad+) cells, wherein the cells are Wharton's jelly mesenchymal stem cells (WJMSCs) and the N-cad+ cells are N-cad+ WJMSCs; and
introducing the N-cad+ cells into a joint space of the subject.

23. The method of claim 22, wherein the joint disease is osteoarthritis (OA).

24. The method of claim 22, further comprising injecting the N-cad+ cells into an intraarticular space of the subject.

25. The method of claim 22, further comprising injecting the N-cad+ cells into the subject via at least two-angle injections that are at an angle with respect to each other.

26. The method of claim 25, further comprising:
injecting a first injection of the N-cad+ cells at a first injection orientation; and
injecting a second injection of the N-cad+ cells at a second injection orientation that is at the angle from the first injection orientation.

27. The method of claim 26, wherein the angle is at least 10 degrees, at least 20 degrees, at least 30 degrees, at least 40 degrees, or at least 45 degrees.

28. The method of one claim 27, wherein the first injection orientation is about normal to about 75 degrees from normal or from horizontal.

29. The method of claim 22, comprising combining the N-cad+ cells into a hydrogel prior to the introducing of the cells into the subject.

30. The method of claim 29, wherein the hydrogel includes a gel percentage ranging from about 40% to about 60%.

31. The method of claim 22, wherein the N-cad+ cells are present at least at 500,000 cells per microliter.

32. An isolated cell population prepared by the process of:
providing a sample of cells having N-cad+ cells mixed with N-cad− cells;
dissociating the cells in the sample;
removing dead cells from the dissociated cells to obtain live cells;
incubating the live cells with a binding molecule that binds to N-cadherin; and
selecting N-cad+ cells using the binding molecule to produce the isolated cell population.

33. The isolated cell population of claim 32, wherein the binding molecule is a biotinylated antibody.

34. The isolated cell population of claim 33, wherein the N-cad+ cells are selected by contacting the biotinylated antibody with an anti-biotin substrate.

35. The isolated cell population of claim 32, wherein the isolated cells are MSCs obtained from Wharton's jelly.

36. The isolated cell population of claim 32, wherein the isolated cells are human cells.

37. The isolated cell population of claim 32, wherein the isolated cells produce elevated levels of anti-inflammatory cytokines relative to a native population of cells.

38. The isolated cell population of claim 32, wherein the isolated cells are effective to suppress the expression of catabolic and inflammatory genes in articular cartilage.
